(19) United States
(12) Patent Application Publication
(10) Pub. No.: US 2011/0172627 A1
(43) Pub. Date: Jul. 14, 2011

(54) UNDERGARMENT LINER

(76) Inventor: QUEENCY D. MATEO, San Diego, CA (US)

(21) Appl. No.: 13/051,923

(22) Filed: Mar. 18, 2011

Related U.S. Application Data
(63) Continuation-in-part of application No. 12/772,153, filed on Apr. 30, 2010.
(60) Provisional application No. 61/174,503, filed on May 1, 2009.
(60) Provisional application No. 61/240,306, filed on Sep. 7, 2009.

(51) Int. Cl. A61F 13/58

(52) U.S. Cl. 604/385.04

(57) ABSTRACT

A thin disposable undergarment liner for preventing undergarment stains comprises a nonwoven sheet that includes a top side, a bottom side, and an adhesive element permanently affixed to the bottom side; a peel away paper that covers the adhesive element; and a plurality of securing tabs to further secure the liner to a user's undergarment wherein each tab includes adhesive. The peel away paper is removed to expose the adhesive and the nonwoven sheet is attached to an interior region of the user's undergarment and the securing tabs are attached to exterior regions of the user's undergarment. The nonwoven sheet may be rectangular, triangular, or hourglass-shaped which includes a first portion adapted for use within the user's posterior area, third portion adapted for use within the user's anterior area, and second portion positioned between the first and third portions and adapted for use within the user's crotch area.

This application is a continuation-in-part of and claims priority from the filing date of U.S. patent application Ser. No. 12/772,153, filed Apr. 30, 2010 (which claimed the benefit of priority under 35 U.S.C. § 119 of provisional application Ser. No. 61/174,503 filed May 1, 2009 and provisional application Ser. No. 61/240,306 filed Sep. 7, 2009), the contents of which are hereby incorporated by reference.

### FIELD OF THE INVENTION

The present invention generally relates to hygiene products. More particularly, the present invention relates to undergarment liners that prevent stains on undergarments.

### BACKGROUND

Many people of various age ranges may experience bodily discharges, including light urinary leakage and fecal smears on clothing after defecation from restroom use. Such incidents do not warrant bulky and absorbable material as used in incontinence products or full-fledged diapers, which are inconvenient and noticeable. There are no adequate products that provide a thin, discreet protective barrier that is disposable and is designed to protect undergarments from stains and keep the undergarments clean.

Currently, none of the undergarment liners on the market specifically address stains from occurring, are developed to act as a disposable protective barrier for any article of clothing subject to urinary and/or fecal contact, and are thin enough to provide comfort while being undetectable by others. Those that are available for purchase are designed to primarily maximize fluid absorbency and are categorized as either incontinence products which are widely used by the geriatric population, feminine hygiene products for menstruation use, or as diapers used by infants and toddlers who have not yet developed bladder and bowel control.

None of the undergarment liners on the market are adapted for use by men of any age. Men of all ages and even teenagers are susceptible to chronic underwear staining even if they do not suffer from incontinence problems. The staining of men's underwear may be due to a variety of reasons such as: poor wiping techniques; poor hygiene; excess moisture due to humid climates; athletic and other physical activity causing excessive perspiration; digestive issues such as irritable bowel syndrome which cause frequent bowel movements; skin sensitivity, rashes, and other conditions making adequate wiping painful and impossible at times; or from the climbing of undergarments into the crevice of the user's buttocks. The products on the market are either designed for incontinence issues or for use by women with menstrual cycles. Even panty liners for women cannot be used for men to prevent stains since the dimensions, shapes and configurations of women's panty liners are not suitable for men, nor are they thin and discreet enough to prevent detection and discomfort.

Additionally, the undergarments liners currently on the market are not adapted for use by children. A child may be beyond the age of requiring full-fledged diapers (which absorb more but are consequently thicker, bulkier, more expensive, and noticeable), but may not yet be of the age to have full bladder or bowel control, or who have not yet mastered personal bathroom hygiene. There is often a transition period after children stop using diapers and start using the restroom on their own, where children do not often clean up or “wipe” adequately after urinating or defecating. There are no products that are designed to protect the child's undergarments during this transition period. Additionally, there are some children who may have difficulty controlling bladder and/or bowel functions due to feelings of anxiousness, fear, or excitement or due to not yet mastering complete bladder and/or bowel control.

Accordingly, there is a need in the art for a thin, discreet, and comfortable undergarment liner that is not meant to absorb large amounts of bodily fluids, but rather acts primarily to prevent stains from setting and merging onto any articles of fabric that the user may wear (for example, briefs, boxers, or panties). There is a need in the art for undergarment liners that are adapted for use by men and teenage boys to prevent staining of their undergarments. Furthermore, there is also a need in the art for an undergarment liner intended for use by children who stain their undergarments although they have outgrown diapers and know how to use restroom facilities. A product that addresses these needs in the art would allow the clothing or underwear to remain clean, retain its original color, and prevents the material from breaking down and disintegrating from the acidity from urine or fecal matter. Such a product would be designed to be thrown away after use, so the more expensive undergarments do not have to be.

### SUMMARY

The present invention provides a undergarment liner that is flexible, and its thin, comfortable profile is designed to protect a user's clothing and undergarments from stains. The present invention is directed to an undergarment liner that is discreet, sleek and resilient, has a soft, cloth-like feel, and can be securely attached onto the user's undergarment to prevent repositioning (and possible detection and discomfort) during athletic or other physical activity. It is difficult for a user to feel the difference between their underwear material and these undergarment liners which allows the user to feel as if they were not using any protection at all. Furthermore, the undergarment liner of the present invention is easy to apply and remove; and it allows a user to simply dispose of unwanted evidence or stained product. By preventing stains on underwear and pants, the present invention assists to prolong the life of clothing material. Getting rid of any type of stains may involve an extended amount of time and chemical detergents and manual friction on focal areas where the stain is concentrated. This product allows the user to bypass these steps, thus saving time on hand washing needs. Additionally, the present invention helps prevent cross contamination of soiled laundry by blocking the user's undergarments from absorbing fecal or urinary material and the likelihood of transferring feces or urine onto other clothes that are simultaneously being washed.

This undergarment liner is absorbent, soft, and sterile on one side, and includes adhesive on the other side. It is applied to the inside of an undergarment, as for example, on the underside and rear regions of the undergarments, where fecal leakage or staining may occur. One embodiment of the present invention is directed to an undergarment liner adapted for use by men to prevent undergarment stains and is comprised of appropriate form, shape, and dimensions to be applied to the inside of a man's undergarment. Another embodiment of the present invention is directed to an undergarment liner adapted for use by children to prevent undergarment stains and is designed in appropriate form, shape, and dimensions to be applied to the inside of children's undergarments (e.g., briefs or boxers for boys or panties for girls). In both embodiments, the present invention may also extend or be positioned toward the front area of the user, where urinary leakage may occur as well. It is designed to protect the undergarment from stains resulting from small urinary or fecal discharge, or from inadequate cleanup or wiping after restroom use. Additionally, in both embodiments, the present invention may further include a plurality of securing tabs to further secure the liner to the user's undergarment to prevent movement of the liner during a man's athletic or strenuous activity (in embodiments of the invention adapted for use by men) or during a child's hyperactivity (in embodiments of the invention adapted for use by children). Nonetheless, the present invention is designed to be relatively inexpensive and disposable. Additionally, the liner is designed to be discreet and non-bulky, so that observers would not notice that a man is wearing an underwear liner in embodiments adapted for use by men thereby avoiding embarrassment or that a child is wearing an undergarment liner under their clothing which is important for developing children to gain confidence, to feel like “big kids” and to avoid ridicule.

To achieve the foregoing and in accordance with the purposes of the present invention, the present invention is directed to a disposable undergarment liner for preventing undergarment stains and is comprised of (a) a nonwoven sheet that includes a top side, a bottom side, and an adhesive element permanently affixed to the bottom side; and (b) a peel away paper that covers the adhesive element wherein the peel away paper includes a glossy side removeably attached to the adhesive element and a matte side that faces opposite the top side of the sheet. In some embodiments of the present invention, the undergarment liner may also include a plurality of securing tabs to further secure the liner to a user's undergarment wherein each tab includes adhesive for attachment of the tab to exterior portions of a user's undergarment. The undergarment liner of the present invention is secured to a user's undergarment by removing the peel away paper to expose the adhesive of the liner and adhering the adhesive directly onto the user's undergarment. The adhesive element of the nonwoven sheet would attach to an interior area of the user's undergarment while adhesive of the securing tabs would attach to exterior areas of the user's undergarment.

The nonwoven sheet may be comprised of a rectangular shape, a triangular shape, an oval shape, or any other shape suitable to adequately address the staining of undergarments. In other embodiments, the nonwoven sheet further comprises a first portion, a second portion, and a third portion wherein the second portion is positioned between the first portion and the third portion, and the second portion is narrower than the first portion and the third portion. The first portion is adapted to be placed in the posterior area of the user's underwear, the second portion is adapted to be placed in the crotch area of the user's underwear, and the third portion is adapted to be placed in the anterior area of the user's underwear. In alternate embodiments, the first portion is adapted to be placed in the anterior area of the user's underwear, the second portion is adapted to be placed in the crotch area of the user's underwear, and the third portion is adapted to be placed in the posterior area of the user's underwear. In some embodiments, the first portion and the third portion are the same shape and dimension thereby making the overall shape of the undergarment liner symmetrical about the vertical and horizontal axis and similar to an hour-glass-like shape which can simultaneously address both urinary and fecal stains in the anterior and posterior areas of the user's undergarment respectively. In such embodiments, the first portion and the third portion may be placed in the posterior area and anterior area of the user's underwear respectively, or the liner may be rotated 180 degrees so that the first portion and the third portion may be placed in the anterior area and posterior area of the user's underwear respectively.

In additional embodiments, the undergarment liner of the present invention may include a plurality of tabs to further secure the liner to a user's undergarment. For example, in one embodiment, the undergarment liner may include: (a) a first securing tab positioned on the center of a first longitudinal side of the nonwoven sheet and (b) a second securing tab positioned on the center of a second longitudinal side of the nonwoven sheet wherein the first securing tab and the second securing tab each includes adhesive for attachment of the securing tabs to exterior regions of a user's undergarment, thereby better securing the undergarment liner to the user's undergarment. In such embodiments, the sheet may be rectangular, oval, or any other suitable shape. In some variations, the first securing tab includes a first elastic component and the second securing tab includes a second elastic component.

In another embodiment in which the nonwoven sheet includes a first portion, second portion, and third portion, the undergarment liner is comprised of: (a) a first securing tab positioned on one longitudinal side of the second portion of the nonwoven sheet and (b) a second securing tab positioned on the opposite longitudinal side of the second portion of the nonwoven sheet. The first securing tab and second securing tab each include adhesive to allow attachment of the undergarment liner to exterior portions of the user's undergarment. In such embodiments, the sheet may be hourglass-shaped, barbell-shaped, or any other suitable shape. In some variations, the first securing tab may include a first elastic component and the second securing tab may include a second elastic component to provide more resilience and comfort for the user. Yet, in another embodiment, the undergarment liner of the present invention may include: (a) a first plurality of securing tabs positioned on one longitudinal side of the second portion of the nonwoven sheet and (b) a second plurality of securing tabs positioned on the opposite longitudinal side of the second portion of the nonwoven sheet wherein each plurality of securing tabs includes two or more securing tabs.

In further embodiments, the nonwoven sheet further comprises a second layer positioned below the top side of the nonwoven sheet and above the bottom side of the nonwoven sheet. This second layer is comprised of a material that is more dense than the nonwoven sheet and provides additional protection for user's with more severe staining issues. In some variations of the invention, the second layer may be comprised of a thin plastic sheet.

One embodiment of the present invention is directed to the above-described disposable undergarment liner for use by men, and such embodiments are thus adapted for use by men and are sized, dimensioned, and shaped accordingly. The embodiments of the invention adapted for use by men mainly address posterior staining of undergarments although some embodiments may also address anterior staining.

Another embodiment of the present invention is directed the above-described disposable undergarment liner for use by children, and such embodiments are thus adapted for use by children and are sized, dimensioned, and shaped accordingly. The embodiments of the invention adapted for use by children can address posterior and/or posterior staining of undergarments. Some variations may address only posterior or anterior staining while other variations address both poster and anterior staining simultaneously. Additionally, other variations of the invention are versatile as such variations may be positioned within the undergarment for either posterior or anterior staining, or they may be positioned within the undergarment to address both posterior and anterior staining simultaneously.

The above description sets forth a summary of embodiments of the present invention so that the description that follows may be better understood and contributions of the present invention to the art may be better appreciated. Some of the embodiments of the present invention may not include all of the features or characteristics listed in the above summary. There may be, of course, other features of the invention that will be described below and may form the subject matter of claims. In this respect, before explaining at least one embodiment of the invention in further detail, it is to be understood that the invention is not limited in its application to the details of the construction and to the arrangement of the components set forth in the following description or as illustrated in the drawings. The invention is capable of other embodiments and of being practiced and carried out in various ways. Furthermore, it is to be understood that the phraseology and terminology employed herein are for the purpose of description and should not be regarded as limiting.

Other features, aspects, and advantages of the present invention will become apparent from the following description of the invention, taken in conjunction with the accompanying drawings, which illustrate, by way of example, various features of embodiments of the invention.

### BRIEF DESCRIPTION OF THE DRAWINGS

FIG. 1 depicts a top view of an undergarment liner in accordance with an embodiment of the present invention.

FIG. 2. depicts a perspective view from below the undergarment liner shown in FIG. I comprising of peel away paper to expose adhesive material in accordance with an embodiment of the present invention.

FIG. 3A depicts a rear view of the undergarment liner shown in FIG. 1 illustrating how the undergarment liner is secured onto the user's underwear in accordance with an embodiment of the present invention.

FIG. 3B depicts a side view of the undergarment liner shown in FIG. 1 illustrating how the undergarment liner is secured onto the user's underwear in accordance with an embodiment of the present invention.

FIG. 4 depicts a rear interior view of the user's pants and illustrates how the undergarment liner shown in FIG. 1 is secured onto the user's pants in accordance with an embodiment of the present invention.

FIG. 5 depicts a magnified transverse cross-sectional view of the undergarment liner shown in FIG. 1 in accordance with an embodiment of the present invention.

FIG. 6 depicts a top view of an undergarment liner in accordance with an embodiment of the present invention.

FIG. 7 depicts a top view of an undergarment liner in accordance with an embodiment of the present invention,

FIG. 8A depicts a top view of an undergarment liner in accordance with an embodiment of the present invention.

FIG. 8B depicts a rear view of underwear with the undergarment liner shown in FIG. 8A therein and illustrates where the undergarment liner is positioned onto the user's underwear in accordance with an embodiment of the present invention.

FIG. 9 depicts a top view of an undergarment liner in accordance with an embodiment of the present invention.

FIG. 10 depicts a top view of an undergarment liner in accordance with an embodiment of the present invention.

FIG. 11 depicts a top view of an undergarment liner in accordance with an embodiment of the present invention.

FIG. 12 depicts a top view of an undergarment liner in accordance with an embodiment of the present invention.

FIG. 13 depicts a top view of an alternate embodiment of the undergarment liner shown in FIG. 6.

FIG. 14 depicts a top view of an alternate embodiment of the undergarment liner shown in FIG. 13.

FIG. 15 depicts a top view of an alternate embodiment of the undergarment liner shown in FIG. 6.

FIG. 16 depicts a top view of an alternate embodiment of the undergarment liner shown in FIG. 15.

### DESCRIPTION OF THE INVENTION

In the following description of embodiments of the invention, reference is made to the accompanying drawings, which form a part of this application. The drawings show, by way of illustration, certain embodiments in which the invention may be practiced. It is to be understood that other embodiments may be utilized and modifications may be made without departing from the scope of the present invention. Also, it is to be understood that the phraseology and terminology employed herein are for the purpose of description and should not be regarded as limiting.

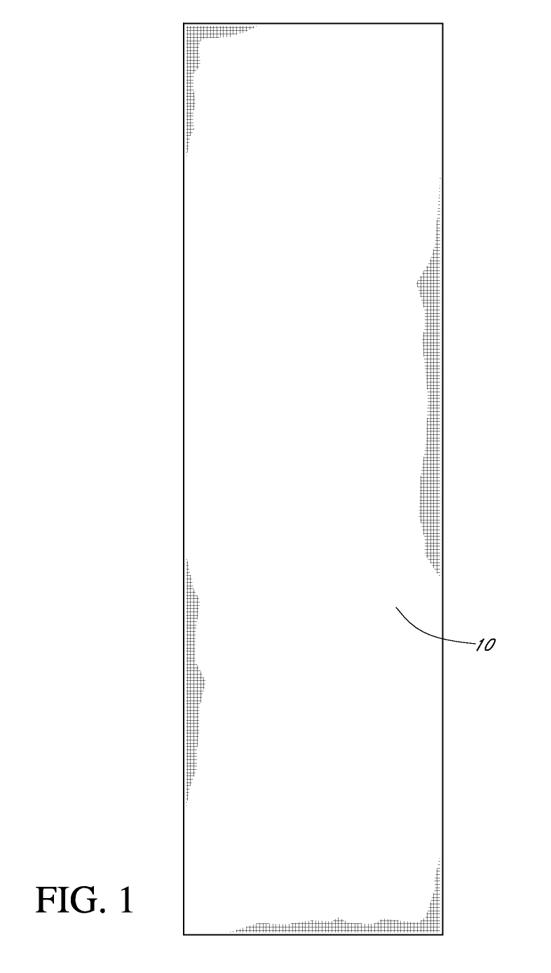

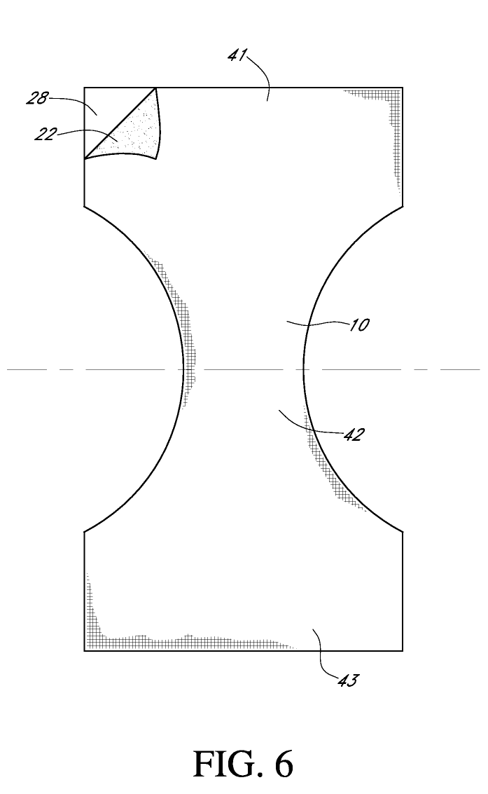

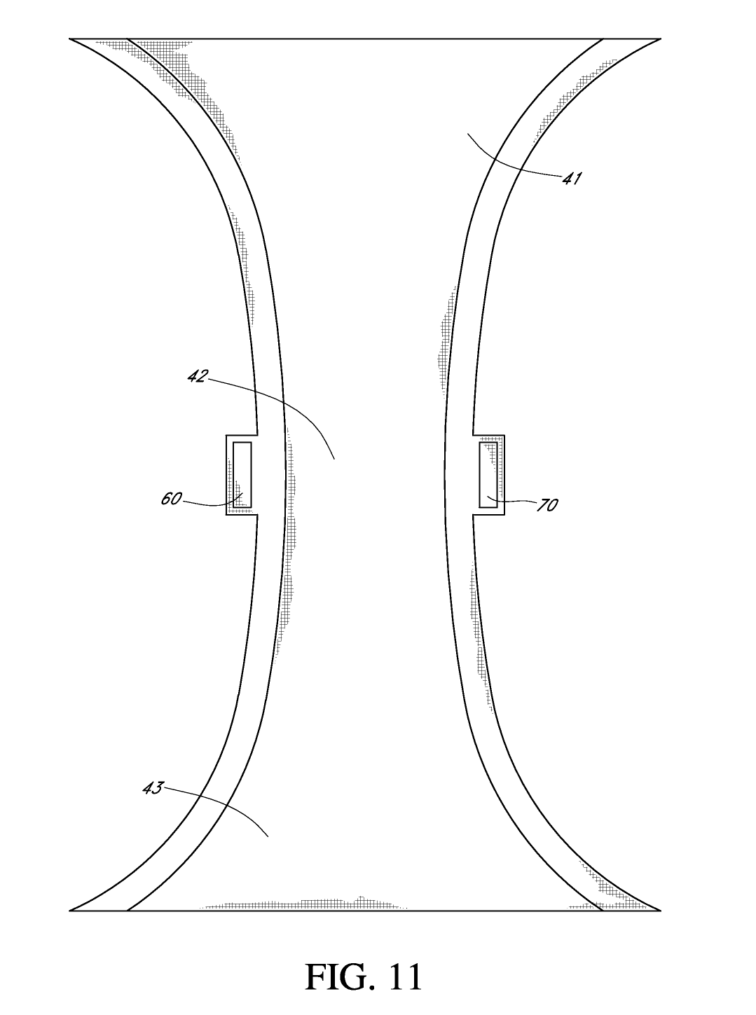

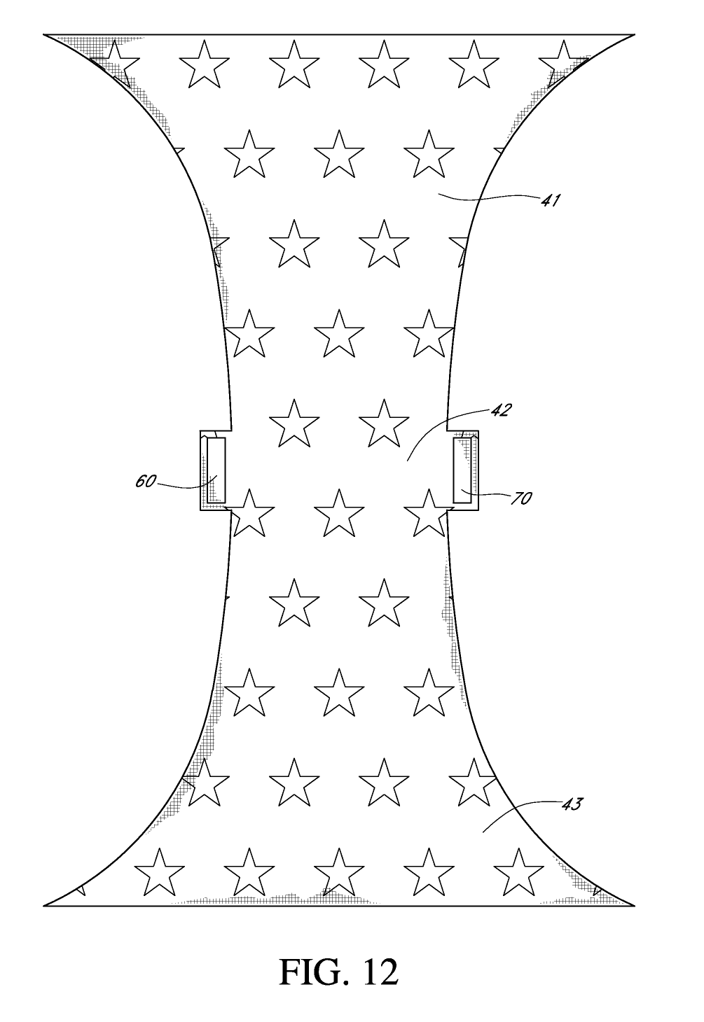

The embodiment of the present invention depicted in FIG. 1 is directed to an undergarment liner that generally is comprised of a sheet 10. In the embodiment shown in FIG. 1, sheet 10 is generally of a rectangular form although it may be comprised of a variety of shapes as exemplified in FIGS. 6-16. Sheet 10 is thin and in many embodiments of the present invention, sheet 10 is as thin as a sheet of paper or as thin as an adhesive bandage such as a BAND-AID®. Sheet 10 is made of a sterile, soft, and absorbent material. It is designed to protect the garment that it is covering from staining caused by mild urinary or fecal discharge.

In most embodiments of the invention, sheet 10 is made of a nonwoven fabric which is generally a sheet or web structures bonded together by entangling fiber or filaments mechanically, chemically, thermally or by solvent treatment. Since the present invention provides a liner that is disposable after each use, sheet 10 being comprised of nonwoven fabrics is ideal since such fabrics may be engineered and designed to have a limited life suitable as a single-use fabric. Additionally, nonwoven fabrics can provide specific functions such as absorbency, resilience, stretch, strength, and/or softness and are suitable as a bacterial barrier to provide sterility. Such properties may be combined to create an appropriate material for sheet 10 that is suited for stain prevention and as a barrier between the user and fecal or urine elements, while achieving a good balance between product use, life, and cost.

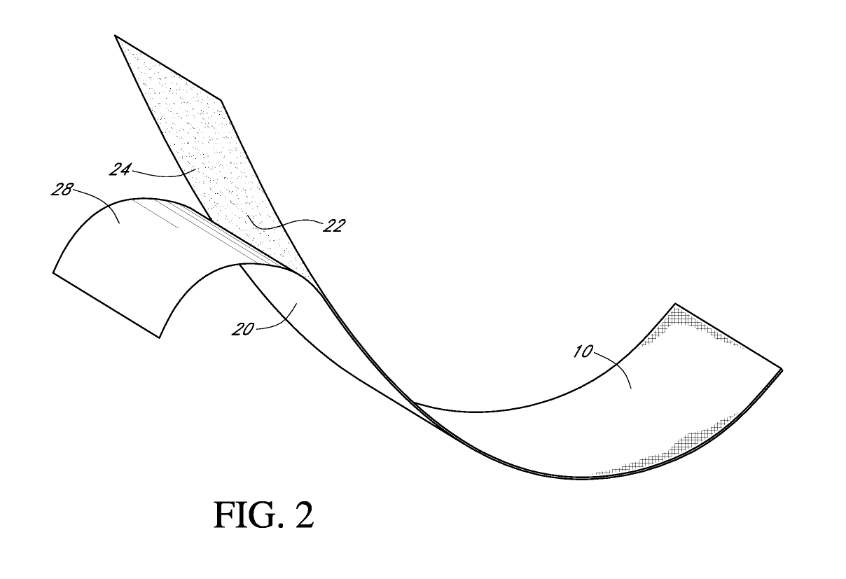

FIG. 2 depicts a perspective view from below the undergarment liner shown in FIG. 1. As shown in FIG. 2, the undergarment liner of the present invention is comprised of: sheet 10 that includes a bottom side 24, an adhesive element 22 that is permanently affixed to bottom side 24, and a peel away paper that covers adhesive element 22. The peel away paper includes a glossy side 28 and a matte side 20. Matte side 20 faces opposite the top side of sheet 10. Glossy side 28 is removeably attached to adhesive element 22 to allow the peel away paper to be removed from adhesive element 22 to expose adhesive element 22 for securing the undergarment liner to an undergarment. When a user wants to secure the undergarment liner to an undergarment, the user would simply remove the peel away to expose adhesive element 22 and the undergarment liner is then adhered directly onto an interior region of the user's undergarment via adhesive element 22. This interior region of the user's undergarment would be the problem area for which the user seeks to protect from urinary and/or fecal staining.

Adhesive element 22 may be comprised of any suitable adhesive known in the art such as hot melt glue, and it may be applied to the entire surface of bottom side 24 or to one or more portions of bottom side 24 in any pattern, configuration, and quantity that is sufficient to secure sheet 10 to the user's undergarment. For example, adhesive element 22 may be comprised of a series of adhesive lines and/or adhesive dots applied to bottom side 24, or adhesive element 22 may be comprised of a single line or strip of adhesive. Adhesive 22 is permanently affixed to bottom side 24 of sheet 10 so when a user removes the undergarment liner from his undergarment, the adhesive is durable enough not to separate from bottom side 24 of sheet 10 and does not remain on the undergarment. Adhesive 22′s adhesive strength is strong enough to be permanently affixed to bottom side 24 and to securely attach sheet 10 to undergarments, but not too strong to allow the liner to be easily removed from the undergarment. The peel away paper is comprised of glossy side 28 and matte side 20; and FIG. 2 illustrates the peel away paper partially peeled from sheet 10 to expose glossy side 28 that is removeably attached to adhesive element 22 and matte side 20 that faces away from sheet 10. Once this peel away paper is removed and adhesive element 22 is exposed, sheet 10 may be attached to the inside of the user's undergarments as a liner to prevent stains.

In the embodiment shown in FIG. 2, the peel away paper is the same shape and dimension of sheet 10 although in other embodiments, the peel away paper may be a different shape or the same shape as sheet 10, and it may be larger or smaller than sheet 10. If the peel away paper is smaller than sheet 10, adhesive element 22 would be applied to only a portion of sheet 10, and the dimension and shape of the peel away paper would correspond with the dimension and shape of adhesive element 22 since the peel away paper functions to protect and maintain the stickiness of adhesive element 22 until sheet 10 is applied to a user's undergarment.

In an alternate embodiment, sheet 10 may further include a second layer that is positioned below the top side or surface of sheet 10 and above bottom side 24. The second layer would provide additional protection from stains and bodily fluids. The second layer may be made of a material that is more dense than the material of sheet 10 and may be made of a material that is closely woven. For instance, the second layer may be comprised of a thin plastic sheet or any other material that is dense enough to prevent stains and bodily fluids from leaking through and onto the undergarment if sheet 10 fails to do its job. Such embodiments that include a second layer would be suitable for users who suffer from more serious stain problems or for users who are experiencing temporary conditions such as frequent bowel movements caused by food poisoning and who desire that their liner be undetectable by others to avoid embarrassment.

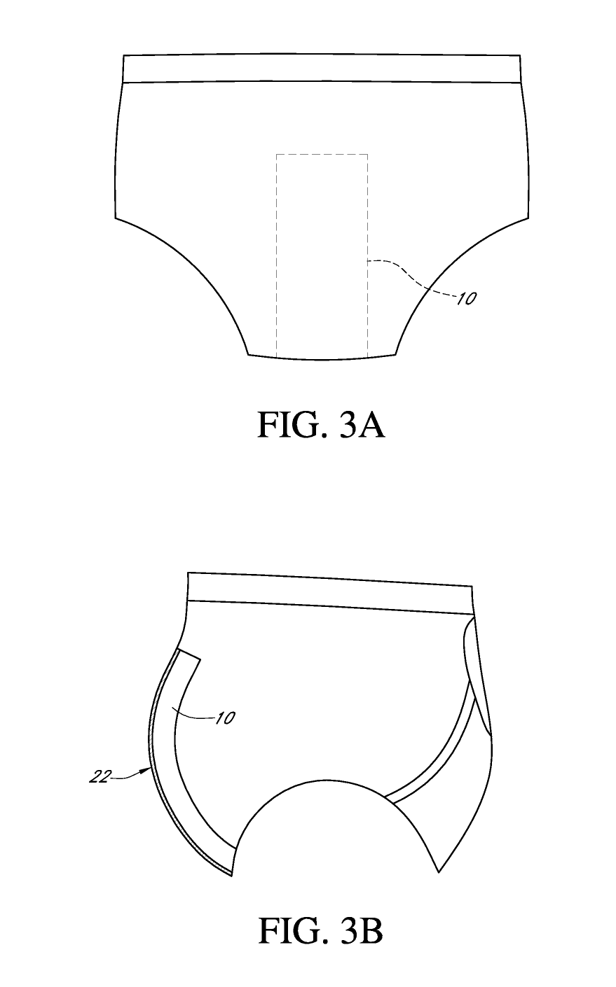

FIG. 3A depicts a method for which the present invention is positioned on a user's undergarment. Sheet 10 is positioned in the middle and lengthwise to parallel the length of the left and right gluteus maximus crevice. FIG. 3B depicts a side view of the undergarment liner positioned to the rear or posterior region of a user's brief so that the undergarment liner contours the user's gluteus maximus crevice. The surface of sheet 10 engages with the crevice of the user's buttocks wherein the undergarment liner is attached to the interior posterior region of the user's undergarment via adhesive element 22. Adhesive element 22 directly adheres to the interior posterior region of the user's undergarment as depicted in FIG. 3B. Alternatively, for those who tend to have urinary stains instead of fecal stains, the undergarment liner of the present invention may be positioned towards the user's frontal area to cover only the user's anterior area, or it may be positioned to cover both the user's anterior and posterior areas. Different variations of the present invention may be of different shapes and sizes, and may continue underneath from the posterior area to the anterior or front of the user's groin or crotch area (to protect against slight urinary discharge as well). In the embodiments of the invention depicted in FIGS. 6-7 and FIG. 9-16, sheet 10 is designed to cover both the anterior and posterior areas of the user simultaneously thereby providing dual stain prevention from fecal and urinary stains.

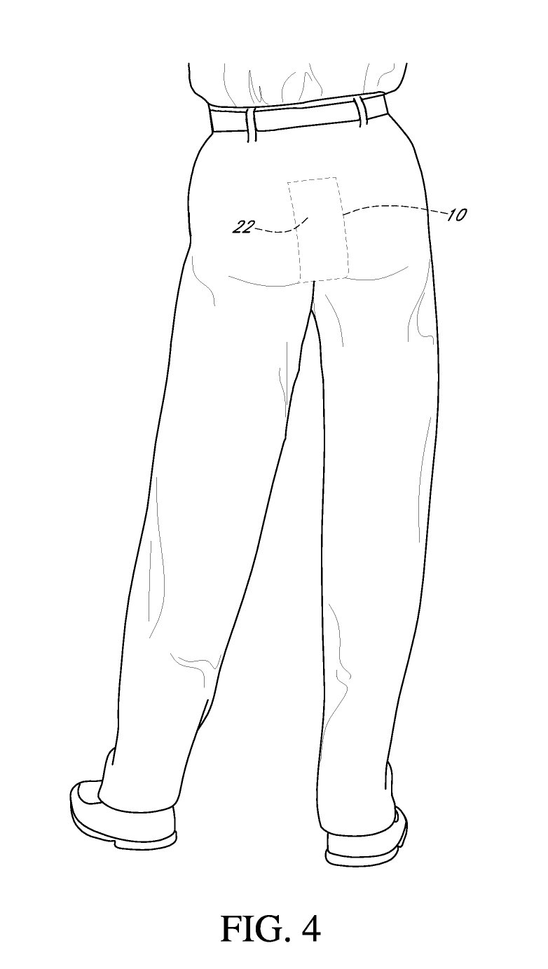

FIG. 4 depicts the application of the present invention inside of a user's pants. Such applications may be suitable for users who prefer not to wear undergarments. The positioning of sheet 10 is secured lengthwise in the middle buttocks area of the user's pants. Adhesive element 22 faces away from the user and directly adheres to the middle buttocks interior area of the pants. For user's who tend to have urinary stains instead of fecal stains, sheet 10 may be positioned towards the user's anterior area instead of user's posterior or buttocks area. Sheet 10 may be placed in any suitable position to address the user's particular staining issues.

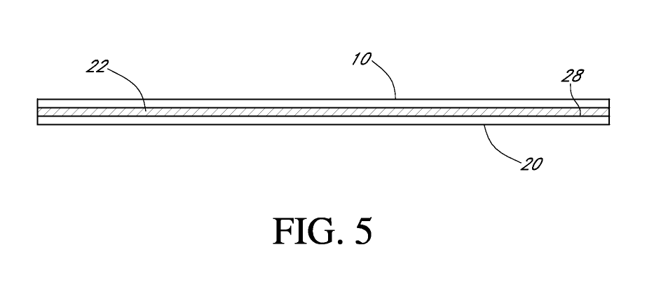

FIG. 5 depicts a magnified transverse cross-sectional view of the undergarment liner of the present invention comprising of sheet 10 which includes adhesive element 22 on bottom side 24 of sheet 10 (see also FIG. 2). As shown in FIG. 5, adhesive element 22 is positioned between sheet 10 and peel away paper that is comprised of matte side 20 and a glossy side 28, wherein glossy side 28 of the peel away paper is removeably attached to adhesive element 22. In the embodiment shown in FIG, 5, the peel away paper covers the entire bottom side 24 of sheet 10 (see also FIG. 2). The surface of sheet 10 comes in direct contact with the user's posterior and/or anterior area when it is secured to the interior of an undergarment and worn by the user. Thus, sheet 10 is preferably made of a thin, comfortable, resilient material such as a nonwoven fabric that provides the user comfort while providing a sufficiently absorbent barrier to prevent stains from setting onto the undergarment.

To use this product, the user (or the parent of the ultimate user in embodiments of the invention adapted for use by children) must remove the peel away paper from sheet 10 and discard in order to expose adhesive element 22 on bottom side 24 of sheet 10. Sheet 10 is then attached to the user's undergarment by directly applying adhesive element 22 onto an interior region of the user's undergarment or article of clothing. The present invention may be applied onto any garment, article of clothing, or undergarment that is directly exposed to a user's skin that is repeatedly exposed to fecal and/or urinary matter (e.g., briefs, boxers, panties, “pull-up” diapers or pants or shorts for those who prefer not to wear underwear). For the embodiment of the invention depicted in FIG. 1, positioning and alignment of sheet 10 usually parallels the crevice separating the buttocks to protect against fecal stains. Alternatively, sheet 10 may be positioned anterior or in front of the groin/crotch area to protect against urinary stains. In some embodiments of the invention in which sheet 10 is long enough to cover both anterior and posterior areas of a user, sheet 10 may be positioned within both the anterior and posterior areas of a user to simultaneously address both fecal and urinary stains .

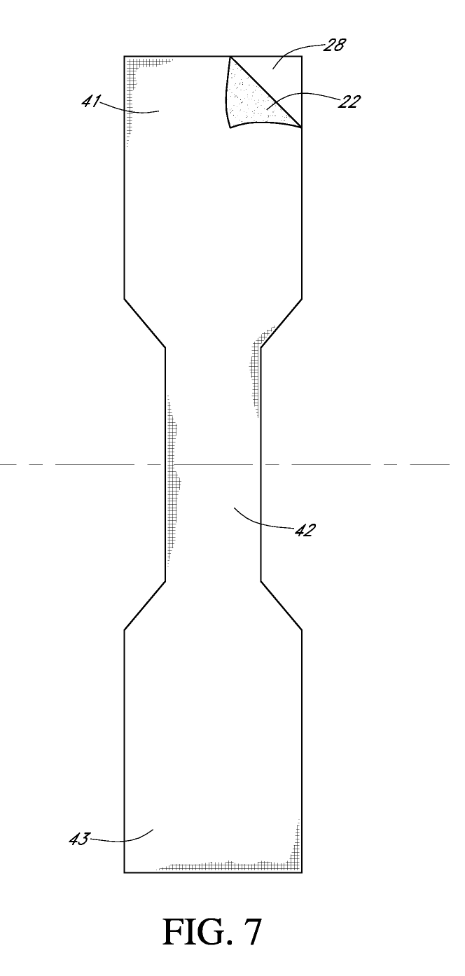

FIGS. 6, 7, and 9-16 depict alternate embodiments of the present invention that address both fecal and urinary stains simultaneously wherein sheet 10 of the undergarment liner comprises: a first portion 41, a second portion 42, and a third portion 43, wherein the first portion 41 and the third portion 43 are connected by the second portion 42. Second portion 42 is positioned between first portion 41 and third portion 43, and second portion 42 is adapted to receive a user's crotch area (i.e., the area of the user adjoining the anterior and posterior regions of the user). In the embodiments of the invention shown in FIGS. 6, 7, and 9-16, second portion 42 is adapted to contour the inner thighs of the user since second portion 42 is narrower than both first portion 41 and third portion 43 thereby creating arches through which the inner thighs of the user pass through. FIGS. 6 and 7 also illustrate adhesive element 22 on the underside of sheet 10 and glossy side 28 of the peel away paper. Glossy side 28 of the peel away paper engages with adhesive element 22 before the peel away paper is removed from sheet 10 before application of the liner to the user's undergarment.

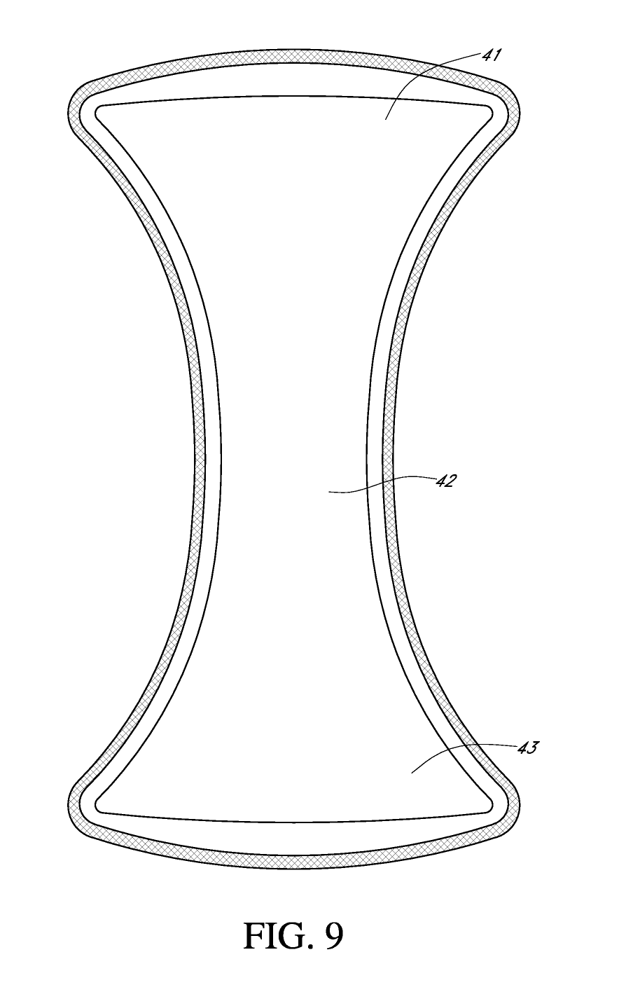

As shown in FIGS. 6, 7, and 9-16, in certain embodiments of the present invention, first portion 41 and third portion 43 are the same shape and size thereby making the overall shape of the undergarment liner symmetrical about the vertical axis and horizontal axis and similar to an hour-glass shape. This hour-glass shape may be of a variety of shapes and sizes. For instance, FIGS. 6 and 9 depict a shape that is relatively wide in the rear and frontal areas (Le., first portion 41 and third portion 43 are wide) which is suitable for larger users (or for users who produce larger stains) and provides more coverage area from stains. On the other hand, FIG. 7 illustrates an undergarment liner shape that has first portion 41 and third portion 43 that is narrower than first portion 41 and third portion 43 of the embodiments of the invention shown in FIGS. 6 and 9. Variations of the invention that include narrow first portion 41 and third portion 43 as shown in FIG. 7 are suitable for smaller users, for users who produce smaller stains, or for users who prefer to wear smaller undergarments such as thongs or G-string underwear as the dimensions and shape of the embodiment shown in FIG. 7 allows the liner to be more discreet from detection.

FIGS. 6, 7 and 9-16 depict an embodiment of the invention adapted for placement within a user's panty ,boxer, brief, or other undergarment as well as any other garment bottoms for users who do not wear undergarments. With its hour-glass-like shape, one section of the undergarment liner may be positioned within the user's rear/posterior area to address fecal stains, and another section of the undergarment liner is positioned within the user's frontal/anterior area to address urinary stains. In one variation of the embodiments shown in FIGS. 6, 7, and 9-16, first portion 41 is adapted to be placed in the posterior area of the user's underwear, second portion 42 is adapted to be placed in the crotch area of the user's underwear, and third portion 43 is adapted to be placed in the anterior area of the user's underwear. Alternatively, in another variation, first portion 41 is adapted to be placed in the anterior area of the user's underwear, second portion 42 is adapted to be placed in the crotch area of the user's underwear, and third portion 43 is adapted to be placed in the posterior area of the user's underwear. First portion 41 and third portion 43 may be sized, shaped and dimensioned to accommodate anterior and posterior regions of certain sexes, sizes, and body types of various users, different user applications or activity intensities, and/or various undergarment types.

In the embodiments shown in FIGS. 6, 7, and 9-16, first portion 41 and third portion 43 are of the same shape and dimension and are thus interchangeable since either first portion 41 or third portion 43 may be used for the rear areas of the user and either first portion 41 or third portion 43 may be used for the frontal areas of the user . For example, first portion 41 may be positioned within the user's rear area, and third portion 43 may be positioned within the user's frontal area. Alternatively, the same undergarment liner may be rotated 180 degrees since placement of the undergarment liner within the user's underwear or article of clothing is not limited to one particular position in such embodiments. As such, after rotation of the undergarment liner, third portion 43 may be positioned within the user's rear area and first portion 41 may be positioned within the user's frontal area.

As depicted in FIGS. 1-5, sheet 10 engages with the user and is the surface that receives any fecal or urinary stains, and glossy side 28 of the peel away paper is initially attached to adhesive element 22 until the peel away paper is peeled away and adhesive element 22 of sheet 10 is applied directly to the interior of the user's undergarment or article of clothing.

In alternate embodiments, wherein sheet 10 is not symmetrical about the horizontal axis, first portion 41 may be larger and/or of a different shape than third portion 43, or first portion 41 may be smaller and/or of a different shape than third portion 43. In some variations of the invention, first portion 41 may be specifically adapted for use with the rear or posterior area of the user, and third portion 43 may be specifically adapted for use with the front or anterior area of the user. Alternatively, first portion 41 may be specifically adapted for use with the front or anterior area of the user, and third portion 43 may be specifically adapted for use with the rear or posterior area of the user.

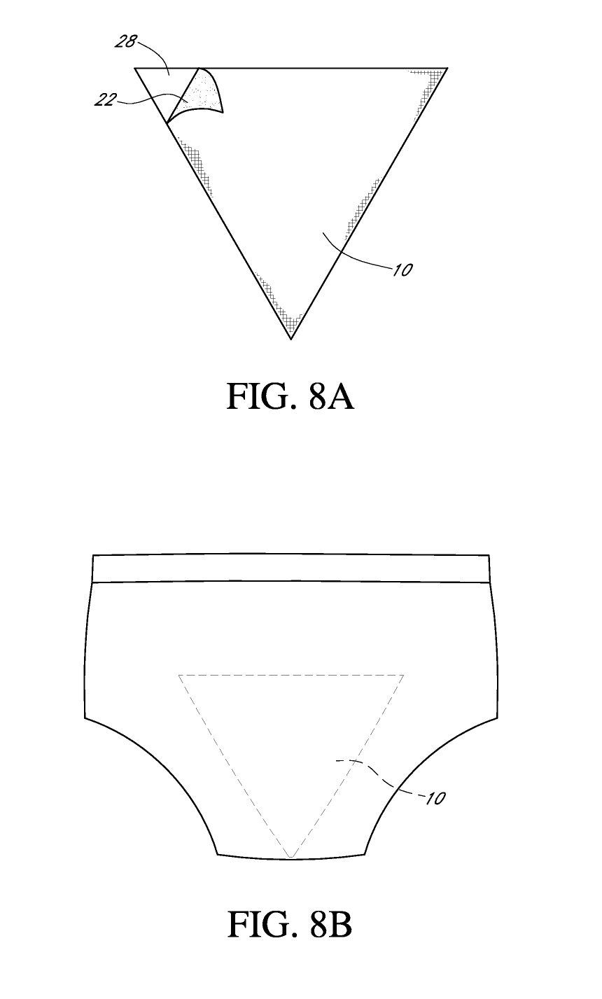

FIGS. 8A and 8B depict another embodiment of the present invention wherein the shape of the undergarment liner is triangular. Referring to FIG. 8A, sheet 10 is triangular in shape and engages with the user as it is the surface that receives any fecal or urinary stains, and glossy side 28 of the peel away paper is initially in contact with adhesive element 22 until it is peeled away and sheet 10 is secured to the undergarment by applying adhesive element 22 onto to an interior region of the user's undergarment or article of clothing. Referring to FIG. 8B, in such triangular-shaped embodiments of the invention, sheet 10 of the undergarment liner may be positioned more towards the rear area of the underwear or article of clothing (to prevent fecal stains) or positioned more towards the frontal area of the underwear or article of clothing (to prevent urinary stains). Accordingly, FIG. 8B may depict either a rear view or a front view of an undergarment with sheet 10 shown in FIG. 8A therein and illustrates how sheet 10 of the undergarment liner is positioned onto an interior region of the user's underwear depending on whether fecal or urinary stains are to be addressed. The triangular-shaped embodiments provide a versatile alternative allowing either fecal or urinary stains to be addressed since some users do not have both fecal and urinary stain issues. If the user does have both fecal and urinary stain issues, then the hour-glass-shaped embodiments illustrated in FIGS. 6,7 and 9-16 would be able to simultaneously address such issues.

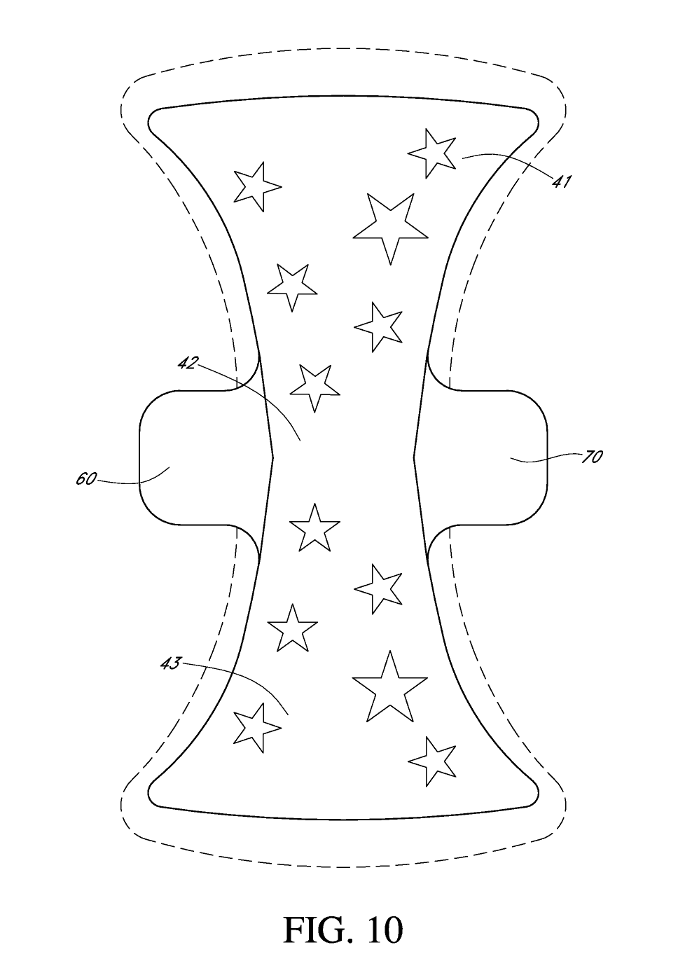

FIGS. 10-16 depict an alternate embodiment of the invention that further includes a plurality of securing tabs to further secure the liner to a user's undergarment. As in the embodiments of the invention depicted in FIGS. 1-9, FIGS. 10-16 illustrate an embodiment of the present invention that also includes: (a) a nonwoven sheet 10 that includes a top side, a bottom side 24, and an adhesive element 22 permanently affixed to bottom side 24 of sheet 10; and (b) a peel away paper that covers adhesive element 22 wherein the peel away paper includes a glossy side 28 removeably attached to adhesive element 22 and a matte side 20 that faces opposite the top side of sheet 10 (see FIGS. 2 and 5). The undergarment liner of the present invention is secured to a user's undergarment by removing the peel away paper to expose adhesive element 22 and adhering adhesive element 22 directly onto an interior region of the user's undergarment. However, the embodiment of the invention shown in FIGS. 10-16 further comprises a plurality of securing tabs to further secure the liner to a user's undergarment wherein each tab includes its own adhesive for attachment of the tab to exterior portions of a user's undergarment. Thus, the adhesive element of the nonwoven sheet would attach to an interior area of the user's undergarment while adhesive elements of the securing tabs would attach to exterior areas of the user's undergarment.

In the embodiment shown in FIGS. 10-12, the undergarment liner includes: (a) a first securing tab 60 positioned on one longitudinal side of second portion 42 of sheet 10 and (b) a second securing tab 70 positioned on the opposite longitudinal side of second portion 42 of sheet 10. First securing tab 60 and second securing tab 70 each include adhesive to allow attachment of the undergarment liner to exterior portions of the user's undergarment thereby better securing the undergarment liner to the user's undergarment. To attach the undergarment liner to an undergarment, the user would remove the peel away paper to expose adhesive element 22 of sheet 10 (see FIGS. 2 and 5) and to expose the adhesive on the undersides of first securing tab 60 and second securing tab 70. The user then would attach adhesive element 22 of sheet 10 directly onto an interior region of the user's undergarment and then fold over both first securing tab 60 and second securing tab 70 so the adhesive of first securing tab 60 and second securing tab 70 can be attached to exterior regions of the user's undergarment.

First securing tab 60 and second securing tab 70 are not limited to a particular dimension and shape as they may be dimensioned and shaped to accommodate any variety of users (of different ages and sizes), applications (e.g., for athletic or other high-level physical activity), or types of undergarments (e.g., large briefs or G-string underwear). Consequently, larger securing tabs may be more suitable for larger users, for athletic activity applications, and/or for larger briefs; while smaller securing tabs may be more suitable for smaller users, for sedentary activity, and/or for smaller underwear varieties such as thong underwear.

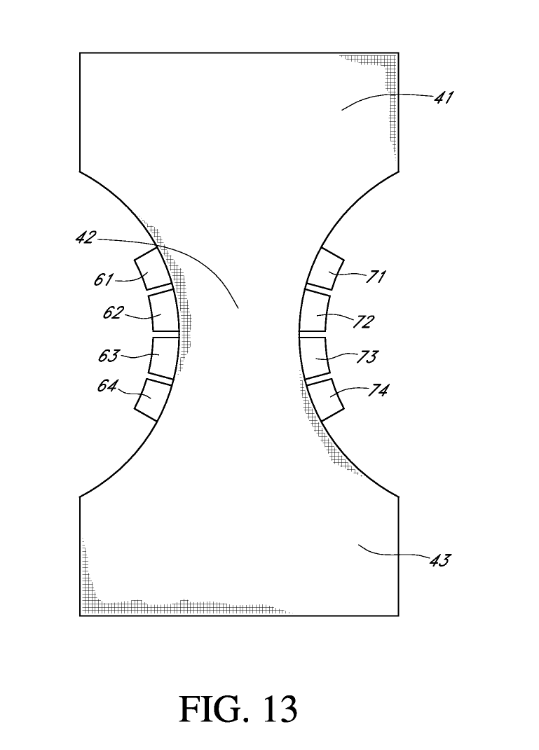

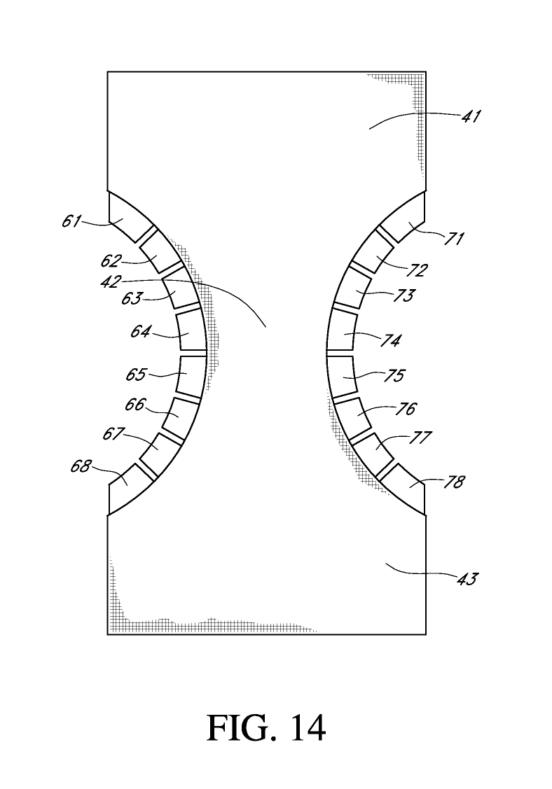

Additionally, the securing tabs are not limited to a specific quantity or a certain configuration. FIGS. 13-14 illustrate an alternate embodiment in which the undergarment liner includes: (a) a first series of securing tabs positioned on one longitudinal side of second portion 42 of sheet 10 and (b) a second series of securing tabs positioned on the opposite longitudinal side of second portion 42 of sheet 10, wherein each series of securing tabs is comprised of a plurality of tabs. In the variation of the invention shown in FIG. 13, the first series of securing tabs is comprised of securing tabs 61, 62, 63, and 64; and the second series of securing tabs is comprised of securing tabs 71, 72, 73, and 74. Securing tabs 61, 62, 63, 64, 71, 72, 73, and 74 would each contain adhesive on the underside. To apply the liner, the user would remove the peel away paper to expose adhesive element 22 of sheet 10 and to expose the adhesive on securing tabs 61, 62, 63, 64, 71, 72, 73, and 74, The user would then attach adhesive element 22 of sheet 10 (see FIGS. 2 and 5) directly onto an interior region of the user's undergarment and then fold over securing tabs 61, 62, 63, 64, 71, 72, 73, and 74 so the adhesive of said securing tabs can be attached to exterior regions of the user's undergarment.

In the embodiment shown in FIG. 13, the first series of securing tabs and second series of securing tabs are positioned within second portion 42 of sheet 10 or within the user's crotch area. However, FIG. 14 depicts another variation of the invention in which portions of the first series of securing tabs and second series of securing tabs approach first portion 41 and third portion 43 of sheet 10.

In the embodiment shown in FIG, 14, the undergarment liner includes: (a) a first series of securing tabs positioned on one longitudinal side of second portion 42 of sheet 10 and (b) a second series of securing tabs positioned on the opposite longitudinal side of second portion 42 of sheet 10. The first series of securing tabs is comprised of securing tabs 61, 62, 63, 64, 65, 66, 67, and 68; and the second series of securing tabs is comprised of securing tabs 71, 72, 73, 74, 75, 76, 77, and 78. Although the first series of securing tabs and second series of securing tabs are positioned substantially within second portion 42 of sheet 10 and run along the entire longitudinal sides of second portion 42, there are portions of the first series of securing tabs and the second series of securing tabs that approach first portion 41 and third portion 43 of sheet 10. For example, tabs 61 and 68 of the first series of securing tabs approach first portion 41 and third portion 43 of sheet 10, respectively; and tabs 71 and 78 of the second series of securing tabs approach first portion 41 and third portion 43 of sheet 10, respectively. The liner would be applied as described above for the embodiment depicted in FIG. 13 with the exception that there are additional securing tabs 65, 66, 67, 68, 75, 76, 77, and 78 to be folded over and attached to the exterior regions of the user's undergarment.

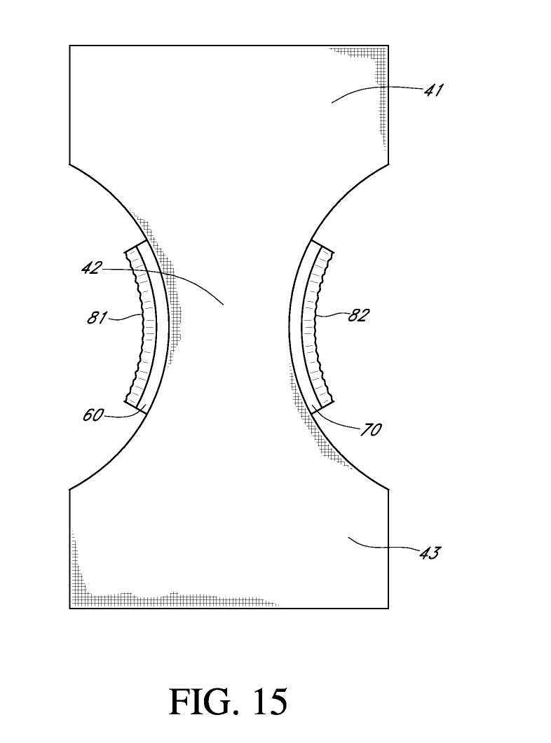

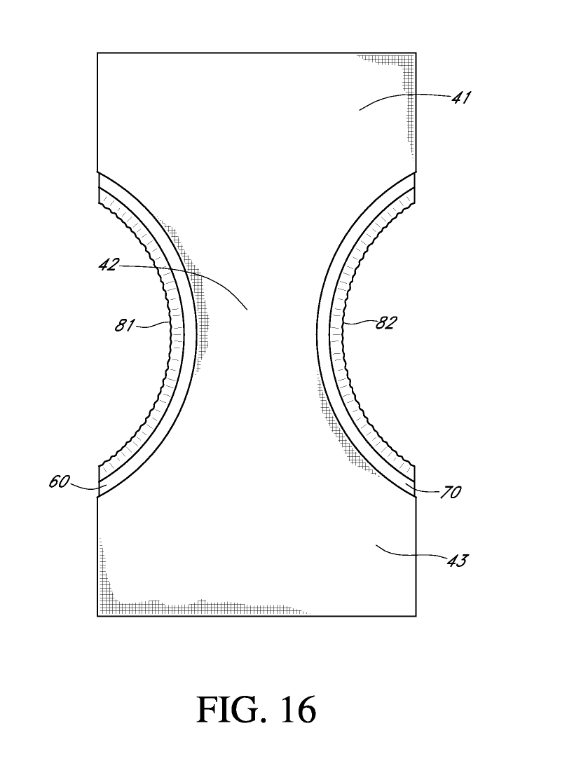

FIGS. 15 and 16 illustrate another embodiment of the present invention in which the undergarment liner is comprised of: (a) a first securing tab 60 positioned on one longitudinal side of second portion 42 of sheet 10, (b) a second securing tab 70 positioned on the opposite longitudinal side of second portion 42 of sheet 10, wherein first securing tab 60 includes a first elastic 81 and second securing tab 70 includes a second elastic 82. First elastic 81 and second elastic 82 provide resilience and flexibility to first securing tab 60 and second securing tab 70 respectively, thereby providing more comfort and security for the user. First elastic 81 and second elastic 82 allow first securing tab 60 and second securing tab 70 to move in accordance with the user's activity without detaching and thus preventing the repositioning or detachment of the liner from the user's undergarment. In the embodiment shown in FIG. 15, first securing tab 60 and second securing tab 70 are substantially positioned along the longitudinal sides of the center of second portion 42 of sheet 10. However, FIG. 16 depicts another embodiment of the invention in which first securing tab 60 and second securing tab 70 are positioned along the entire longitudinal sides of second portion 42 of sheet 10, and first securing tab 60 and second securing tab 70 approach first portion 41 and third portion 43 of sheet 10.

As illustrated in FIGS, 10-16, the quantity of securing tabs is not limited to two (one on each side of the liner) as there may be a plurality of tabs on each side of the liner; nor is the positioning of the securing tabs limited exclusively to second portion 42 of sheet 10. Rectangular-shaped (see e.g., FIGS. 1-3), oval-shaped, or any other suitable shaped variations of the invention may also include a plurality of securing tabs to better secure the liner to the user's undergarment. In such variations, the securing tabs may be positioned substantially along the longitudinal sides of the center area of the liner (or area of the liner that accommodates the user's crotch area) so that the securing tabs can be easily folded over to adhere to exterior regions of the user's undergarment. For example, in one embodiment, the undergarment liner may be comprised of (a) a first securing tab positioned on the center of a first longitudinal side of the sheet and (b) a second securing tab positioned on the center of a second longitudinal side of the sheet wherein the first securing tab and the second securing tab each includes adhesive for attachment of the first securing tab and the second securing tab to exterior regions of a user's undergarment.

Also, there may be any suitable quantity of securing tabs, and they may be configured in a variety of ways. The multiple securing tabs on each side of second portion 42 of sheet 10 allow a more snug fit as each tab can be individually positioned to accommodate a wide variety of undergarments. The positioning of the securing tabs may reside exclusively within second portion 42 of sheet 10, or some of the securing tabs may approach or be positioned within first portion 41 and third portion 43 of sheet 10. Additionally, as discussed above with respect to first securing tab 60 and second securing tab 70 of FIGS. 10-12, the securing tabs utilized in the present invention are not limited to a particular dimension and shape as they may be dimensioned and shaped to accommodate any variety of users (of different ages and sizes), applications (e.g., for athletic or other high-level physical activity), or types of undergarments (e.g., large briefs or G-string underwear).

### Further Embodiments

Although the present invention has been described above with reference to certain versions thereof as depicted in the drawings, other versions are possible. For instance, in addition to being specifically adapted for use by either men or by children, certain embodiments of the invention may be adapted for use by women. Unlike typical panty liners currently on the market, the undergarment liner of the present invention has a larger surface area making it possible for women to also use the liners to address fecal stains or both fecal and urinary stains. The thin, sleek, and discreet features of the liners are ideal for women who have issues with stains but desire to wear thin, tight, or revealing clothes through which normal panty liners would easily be detected.

Variations of the present invention may be of different sizes (appropriate for different sized men, children of different ages, children of different sizes and weights, fitted for different kinds of boys' or girls' undergarments, different thicknesses and absorbencies, different materials, made with different scents, with different types of adhesive backing (perhaps based on the material of the undergarment it is to be applied to or based on the activity of the user), and even adorned with different coloring, logos, or cartoon characters or superhero designs to appeal to children.

In alternate embodiments of the invention, variations can comprise of biodegradable, flushable material, organic material, recycled paper/paper products, paper products, any woven cloth material, or cotton or other similar textile. Sizes can vary, and be greater or less than 8 cm wide×28 cm in length, in order to accommodate for extra width and length to prevent stains from mild urinary discharge, or very small to accommodate children's sizes. Shapes can range anywhere from straight-edged or curved-edged rectangle, square, circular, flattened oval, disk-shaped, semi-circular, parabola-shaped, T-shaped, H-shaped, U-shaped, barbell-shaped, soft curved edges, panty outlined cut-outs or brief style outlined cut-outs to specifically match the shape of the appropriate undergarment, or any other appropriate shape to protect the undergarment from stains while remaining thin enough to provide comfort for the user and to remain undetectable from observers. Adhesive variations can include polyurethane, polyvinyl acetate (PVA's), pressure sensitive adhesives, water based adhesives, vegetable glues, silicone adhesives, film adhesives, or variations thereof. W Child-friendly adhesives may be used, and variations of this product may be designed to be even more simple to attach (for example, with directions and diagrams on the peel away paper itself) in order to promote attachment by children themselves. This invention can be specifically designed for children of all ages and both sexes. The present invention may further include additional structures and features including aesthetically pleasing features such as various designs and colors, various logos, cartoon characters, or superhero figures to appeal to children. It may also be customized or adapted for use by certain user body-types and sizes, and may also include deodorant, antiperspirant, fragrance, activated charcoal, talcum powder, baking soda, or other fragrance-producing or odor-absorbing materials.

While the description above refers to particular embodiments of the present invention, it will be understood that many modifications may be made without departing from the spirit thereof. The presently disclosed embodiments are therefore to be considered in all respects as illustrative and not restrictive.

What is claimed is:

1. A disposable undergarment liner for preventing undergarment stains, the undergarment liner comprising:
a nonwoven sheet comprised of
a top side,
a bottom side, and
an adhesive element permanently affixed to the bottom side;
a peel away paper that covers the adhesive element wherein the peel away paper is comprised of
a glossy side removeably attached to the adhesive element and
a matte side that faces opposite the top side wherein the peel away paper is removed to expose the adhesive element and the adhesive element adheres directly onto an interior region of a user's undergarment;
a first securing tab positioned on the center of a first longitudinal side of the nonwoven sheet; and
a second securing tab positioned on the center of a second longitudinal side of the nonwoven sheet wherein the first securing tab and the second securing tab each includes an adhesive for attachment of the first securing tab and the second securing tab to exterior regions of the user's undergarment.

2. The undergarment liner of claim 1 wherein the nonwoven sheet is comprised of a rectangular shape.

3. The undergarment liner of claim 1 wherein the nonwoven sheet further comprises a first portion, a second portion, and a third portion wherein the second portion is positioned between the first portion and the third portion and the second portion is positioned between the center of the first longitudinal side of the nonwoven sheet and the center of the second longitudinal side of the nonwoven sheet.

4. The undergarment liner of claim 3 wherein the second portion is narrower than the first portion and the third portion.

5. The undergarment liner of claim 4 wherein the nonwoven sheet is comprised of an hourglass shape.

6. The undergarment liner of claim 4 wherein the first portion is adapted to be placed in the posterior area of the user's undergarment, the second portion is adapted to be placed in the crotch area of the user's undergarment, and the third portion is adapted to be placed in the anterior area of the user's undergarment.

7. The undergarment liner of claim 4 wherein the first portion and the third portion are of the same shape and dimension.

8. The undergarment liner of claim 1 wherein the first securing tab includes a first elastic component and the second securing tab includes a second elastic component.

9. The undergarment liner of claim 1 wherein the undergarment liner is adapted for use by men.

10. The undergarment liner of claim 1 wherein the undergarment liner is adapted for use by children.

11. A disposable undergarment liner for preventing undergarment stains, the undergarment liner comprising:
a nonwoven sheet comprised of
a top side,
a bottom side,
an adhesive element permanently affixed to the bottom side;
a first portion,
a second portion, and
a third portion wherein the second portion is positioned between the first portion and the third portion;
a peel away paper that covers the adhesive element wherein the peel away paper is comprised of
a glossy side removeably attached to the adhesive element and
a matte side that faces opposite the top side wherein the peel away paper is removed to expose the adhesive element and the adhesive element adheres directly onto an interior region of a user's undergarment;
a first securing tab positioned on a first longitudinal side of the second portion of the nonwoven sheet; and
a second securing tab positioned on a second longitudinal side of the second portion of the nonwoven sheet wherein the first securing tab and the second securing tab each includes an adhesive for attachment of the first securing tab and the second securing tab to exterior regions of the user's undergarment.

12. The undergarment liner of claim 11 wherein the nonwoven sheet is comprised of a rectangular shape.

13. The undergarment liner of claim 11 wherein the second portion is narrower than the first portion and the third portion.

14. The undergarment liner of claim 13 wherein the nonwoven sheet is comprised of an hourglass shape.

15. The undergarment liner of claim 11 wherein the first portion is adapted to be placed in the posterior area of the user's undergarment, the second portion is adapted to be placed in the crotch area of the user's undergarment, and the third portion is adapted to be placed in the anterior area of the user's undergarment.

16. The undergarment liner of claim 13 wherein the first portion and the third portion are of the same shape and dimension.

17. The undergarment liner of claim 11 wherein the undergarment liner is adapted for use by men.

18. The undergarment liner of claim 11 wherein the undergarment liner is adapted for use by children.

19. A disposable undergarment liner for preventing undergarment stains, the undergarment liner comprising:
a nonwoven sheet comprised of
a top side,
a bottom side,
an adhesive element permanently affixed to the bottom side;
a first portion,
a second portion, and
a third portion wherein the second portion is positioned between the first portion and the third portion;
a peel away paper that covers the adhesive element wherein the peel away paper is comprised of
a glossy side removeably attached to the adhesive element and
a matte side that faces opposite the top side wherein the peel away paper is removed to expose the adhesive element and the adhesive element adheres directly onto an interior region of a user's undergarment;
a first plurality of securing tabs positioned on a first longitudinal side of the second portion of the nonwoven sheet; and
a second plurality of securing tabs positioned on a second longitudinal side of the second portion of the nonwoven sheet wherein the first plurality of securing tabs and the second plurality of securing tabs include an adhesive for attachment of the first plurality of securing tabs and the second plurality of securing tabs to exterior regions of the users undergarment.

20. The undergarment liner of claim 19 wherein the second portion is narrower than the first portion and the third portion.
